(19) United States
(12) Patent Application Publication
(10) Pub. No.: US 2016/0339015 A1
(43) Pub. Date: Nov. 24, 2016

(54) ORAL PHARMACEUTICAL COMPOSITION OF METHYLERGONOVINE

(71) Applicant: Lupin Inc., Somerset, NJ (US)

(72) Inventors: James Garegnani, Somerset, NJ (US); Avinash Nangia, Somerset, NJ (US)

(73) Assignee: Lupin Inc., Somerset, NJ (US)

(21) Appl. No.: 15/160,831

(22) Filed: May 20, 2016

Related U.S. Application Data
(63) Continuation of application No. 62/164,052, filed on May 20, 2015.

(51) Int. Cl. A61K 31/48; A61K 9/48; A61K 9/24; A61K 9/00; A61K 9/20

(52) U.S. Cl. CPC A61K 31/48; A61K 9/0053; A61K 9/20; A61K 9/2072; A61K 9/209; A61K 9/4808; A61K 9/4833

(57) ABSTRACT

An oral modified release pharmaceutical composition of methylergonovine suitable for oral once daily administration is provided. The composition includes at least about 0.6 mg dose of methylergonovine or a pharmaceutically acceptable salt thereof and is particularly useful for treating migraine or refractory migraine.

### CROSS-REFERENCE TO RELATED APPLICATIONS

This application claims the benefit of priority of provisional patent application No. 62/164,052, filed on May 20, 2015, the contents of which are incorporated herein in their entirety.

### BACKGROUND OF THE INVENTION

(a) Field of the Invention

The present invention is directed to an oral modified release pharmaceutical composition of methylergonovine suitable for once daily administration. The composition comprises at least about 0.6 mg dose of methylergonovine and is suitable for once daily administration. The invention is further directed to use of said composition for the treatment of methylergonovine responsive conditions such as migraine, refractory migraine, uterine atony, uterine haemorrhage, subinvolution of the uterus, and uterine hemorrhage in the second stage of labor. Particularly, the present invention provides a method of using said composition for treating migraine or refractory migraine.

(b) Description of the Related Art

Methylergonovine is a semisynthetic analogue of ergonovine, a psychedelic alkaloid found in ergot, and many species of morning glory. The marketed products contain the maleate salt of methylergonovine. Methylergonovine maleate is an active metabolite of methysergide, a drug well known for its value in migraine and cluster headache prevention but which is currently not available. Methylergonovine is a smooth muscle constrictor that mostly is used as a uterine contraction drug. It is most commonly used to prevent or control excessive bleeding following childbirth and spontaneous or elective abortion, and also to aid in expulsion of retained products of conception after a missed abortion (miscarriage in which all or part of the foetus remains in the uterus) and to help deliver the placenta after childbirth.

Methylergonovine is available as injection (intramuscular or intravenous) or as liquid and tablets to be taken orally. The molecular formula of methylergonovine maleate is C₂₀H₂₅N₃O₂.C₄H₄O₄, and its structural formula is:

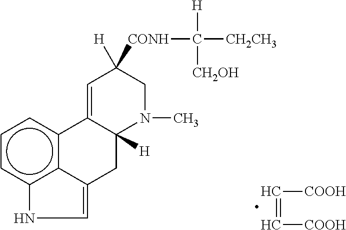

Currently, tablet and injection dosage forms of methylergonovine maleate are available in the United States under as the brand Methergine® from Novartis. Methergine® tablets are available in the 0.2 mg strength and Methergine® injection is available in 0.1 mg and 0.2 mg strengths. For effective treatment of uterine atony, haemorrhage and subinvolution of the uterus, and control of uterine haemorrhage and as per the administration recommendation, the tablets are administered 3-4 times daily and injection is administered at intervals of 2-4 hours.

Due to its vasoconstriction properties, Methergine® is also recommended for several situations as related to some headache patients. Particularly, it is recommended for treating vascular headaches, such as migraine (including menstrual migraine) or cluster.

Migraine headaches are a potentially chronic, progressive and pervasive disease that erodes the sufferer's daily quality of life as well as substantially affecting patients' families, workplaces and society. Historically, migraine has been characterized as episodic attacks separated by normal, symptom-free periods. Findings from migraine sufferers surveyed in a national poll revealed that migraineurs do not view their migraines as isolated events. These sufferers consider their migraines part of a cycle of suffering, treating their current attack and worrying about when the next attack will strike. (J. L. Brandes, Headache: The Journal of Head and Face Pain; Vol. 48, p. 430-441, March 2008).

Effect of methylergonovine in treating refractory migraine has been also studied. Graff-Radford and Bittar reported on 60 consecutive patients with what the authors called drug-induced refractory headache; the authors suggested that methylergonovine was effective in 73% of the patients (S B Graff-Radford, G T Bittar; The use of methylergonovine in the initial control of drug-induced refractory headache; Headache, 1993; 33 (7): 390-393).

The role of methylergonovine in treating refractory migraine has been recently evaluated by Saper and Evans (Joel R. Saper, Randolph W. Evans; Oral methylergonovine maleate for refractory migraine and cluster headache prevention; Headache, 2013; 53(2): 378-381).

There is no standard definition of refractory migraine available and the condition is judged on the basis of symptoms. Refractory migraine is understood to describe a variety of clinical symptoms associated with persistent headache that is difficult to treat or fails to respond to standard headache treatments.

The currently available oral dosage forms of methylergonovine require multiple daily administration for treatment. Particularly, tablets containing 0.2 mg to 0.4 mg of methylergonovine are required to be administered three times daily, and in some cases four times daily administration is suggested to narrow the interval between dosing. In the case of chronic migraine treatment, patients are given a first dose of methylergonovine maleate followed by further multiple doses until the patient experiences relief from pain. The need of such frequent administration of currently available products, however, may result in less preferred treatment choices for many patients, mainly due to compliance issues and especially in case of geriatric patients.

Further, there are adverse effects associated with currently available products and their dosing regimen. The most common adverse effect is hypertension associated in several cases with seizure and/or headache. Hypotension has also been reported. Abdominal pain (caused by uterine contractions), nausea and vomiting have occurred occasionally.

There remains a need for an oral composition containing a unique dose and an improved formulation to deliver methylergonovine. The composition advantageously needs to be administered once per day and provide round-the-clock management of methylergonovine responsive conditions, including migraine and refractory migraine. Such composition should also provide equal or more therapeutic benefits than those achieved by multiple administrations of currently known 0.2 mg methylergonovine dosage forms.

### SUMMARY OF THE INVENTION

The present invention provides an oral modified release pharmaceutical composition of methylergonovine suitable for once daily administration.

In one aspect, the invention provides an oral modified release pharmaceutical composition suitable for once daily administration comprising at least about 0.6 mg of methylergonovine or a pharmaceutically acceptable salt thereof.

In another aspect, the invention provides an oral modified release pharmaceutical composition suitable for once daily administration comprising about 0.6 mg, about 0.8 mg, about 1 mg, about 1.2 mg, about 1.4 mg, about 1.6 mg, about 1.8 mg or about 2 mg of methylergonovine or a pharmaceutically acceptable salt thereof, as well as fractional values between these stated values.

In another aspect, the invention provides an oral modified release pharmaceutical composition suitable for once daily administration comprising at least about 0.6 mg methylergonovine or a pharmaceutically acceptable salt thereof, wherein methylergonovine or a pharmaceutically acceptable salt thereof in the composition is released from the composition over a period ranging from about 0 hour to up to about 24 hours.

In another aspect, the invention provides an oral modified release pharmaceutical composition suitable for once daily administration comprising at least about 0.6 mg methylergonovine or a pharmaceutically acceptable salt thereof, wherein methylergonovine or a pharmaceutically acceptable salt thereof in the composition is released in an extended release manner, with or without an initial load dose.

In another aspect, the invention provides an oral modified release pharmaceutical composition comprising:
(a) at least one extended release portion comprising methylergonovine or a pharmaceutically acceptable salt thereof, and
(b) optionally, at least one immediate release portion comprising methylergonovine or a pharmaceutically acceptable salt thereof.

In another aspect, the invention provides an oral modified release pharmaceutical composition suitable for once daily administration comprising:
(a) a core comprising at least about 0.4 mg to about 1.6 mg methylergonovine or a pharmaceutically acceptable salt thereof exhibiting extended release, and
(b) at least one layer surrounding the core comprising at least about 0.2 mg to about 0.6 mg methylergonovine or a pharmaceutically acceptable salt thereof exhibiting immediate release.

In another aspect, the invention provides an oral modified release pharmaceutical composition suitable for once daily administration comprising:
(a) at least one immediate release portion comprising at least about 0.2 mg to about 0.6 mg methylergonovine or a pharmaceutically acceptable salt thereof, and
(b) at least one extended release portion comprising about 0.4 mg to about 1.6 mg methylergonovine or a pharmaceutically acceptable salt thereof.

In another aspect, the immediate release portion of the composition exhibits complete release of methylergonovine or a pharmaceutically acceptable salt thereof within about 1 hour after oral administration.

In another aspect, the extended release portion of the composition exhibits release of methylergonovine or a pharmaceutically acceptable salt thereof over a period of up to about 24 hours after oral administration.

In another aspect, the release of methylergonovine or pharmaceutically acceptable salt thereof from the extended release portion of the composition starts about 2 hours, about 4 hours, about 6 hours or about 8 hours after oral administration.

In another aspect, the invention provides an oral modified release pharmaceutical composition suitable for once daily administration comprising:
(a) a core comprising at least about 0.4 mg to about 1.6 mg of methylergonovine or a pharmaceutically acceptable salt thereof exhibiting extended release, and
(b) at least one layer surrounding the core comprising at least about 0.2 mg to about 0.6 mg of methylergonovine or a pharmaceutically acceptable salt thereof exhibiting immediate release.

In another aspect, the invention provides a solid oral unit dosage form suitable for once daily administration comprising:
(a) at least one layer comprising least about 0.2 mg to about 0.6 mg methylergonovine or a pharmaceutically acceptable salt thereof exhibiting immediate release, and
(b) at least one layer comprising about 0.4 mg to about 1.6 mg methylergonovine or a pharmaceutically acceptable salt thereof exhibiting extended release.

In another aspect, the invention provides a method of treating a condition selected from migraine, refractory migraine, uterine atony, uterine haemorrhage, subinvolution of the uterus, or uterine haemorrhage in the second stage of labor. The method comprises once daily oral administration of a modified release pharmaceutical composition comprising at least about 0.6 mg dose of methylergonovine or pharmaceutically acceptable salt thereof.

In another aspect, the invention provides a method of treating migraine or refractory migraine. The method comprises once daily oral administration of a modified release pharmaceutical composition comprising at least about 0.6 mg dose of methylergonovine or pharmaceutically acceptable salt thereof.

In another aspect, the invention provides a method for the treatment of refractory migraine comprising orally administering to a patient in need thereof the modified release pharmaceutical composition as described herein.

Still other aspects and advantages of the invention will be apparent from the following detailed description of the invention.

### DETAILED DESCRIPTION OF THE INVENTION

The invention provides an oral modified release pharmaceutical composition suitable for once daily administration of methylergonovine or a pharmaceutically acceptable salt thereof. Preferably, the composition contains at least about 0.6 mg of methylergonovine or a pharmaceutically acceptable salt thereof, and preferably the maleate salt thereof.

The invention addresses the need for a composition of methylergonovine which requires only once daily administration and provides relief which is comparable to that achieved by single as well as multiple administrations of currently available methylergonovine maleate 0.2 mg tablets, including Methergine®.

A once daily administration of methylergonovine is advantageous over a multiple administration dosing regimen in terms of both patient compliance and potential reduction in adverse events associated with the drug, thereby providing better treatment of the conditions for which methylergonovine chloride or methylergonovine maleate is indicated.

The inventors have devised a unique oral modified release pharmaceutical composition in order to provide for an effective once daily form of methylergonovine that may provide round-the-clock desired therapeutic effects while minimizing, if not eliminating, the undesired side effects.

The oral modified release pharmaceutical composition of the invention comprises at least about 0.6 mg methylergonovine or a pharmaceutically acceptable salt thereof and is suitable for once daily administration.

The term “pharmaceutically acceptable salt” as used herein refers to salts which are known to be non-toxic and are commonly used in the pharmaceutical literature. Typical inorganic acids used to form such salts include hydrochloric, hydrobromic, hydroiodic, nitric, sulfuric, phosphoric, hypophosphoric, and the like. Salts derived from organic acids, such as aliphatic mono and dicarboxylic acids (e.g. maleate, tartrate), phenylsubstituted alkanoic acids, hydroxyalkanoic and hydroxyalkandioic acids, aromatic acids, aliphatic and aromatic sulfonic acids, may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methyl benzoate, o-acetoxybenzoate, naphthalene-2-benzoate, bromide, isobutyrate, phenylbutyrate, beta-hydroxybutyrate, chloride, cinnamate, citrate, formate, fumarate, glycolate, heptanoate, lactate, maleate, hydroxymaleate, malonate, mesylate, nitrate, oxalate, phthalate, phosphate, monohydro genphosphate, dihydrogenphosphate, metaphosphate, pyrophosphate, propionate, phenylpropionate, salicylate, succinate, sulfate, bisulfate, pyrosulfate, sulfite, bisulfite, sulfonate, benzenesulfonate, p-bromophenylsulfonate, chlorobenzenesulfonate, ethanesulfonate, 2-hydroxyethanesulfonate, methanesulfonate, naphthalene-1-sulfonate, naphthalene-2-sulfonate, p-toluenesulfonate, xylenesulfonate, tartarate, and the like. A preferred salt is the maleate salt.

The term “modified release” as used herein in relation to the composition according to the invention means release, which is not immediate release as such and is taken to encompass controlled release, sustained release, prolonged release, timed release, retarded release, extended release and delayed release or combinations thereof with immediate release. The term “modified release dosage form” as used herein can be described as dosage forms whose drug-release characteristics of time course and/or location are chosen to accomplish therapeutic or convenience objectives not offered by conventional dosage forms such as a solution or an immediate release dosage form. Modified release solid oral dosage forms include both delayed and extended release drug products (as per US FDA guideline for ‘SUPAC-MR: Modified Release Solid Oral Dosage Forms’).

The “immediate release” refers to the release of the active ingredient over a period of time less than 1 hour after initiation of the release.

As used herein, the term “extended release” refers to the release of the active ingredient over an extended period of time leading to relatively lower peak plasma concentrations and a prolonged bioavailability as compared to “immediate release” compositions of the same active ingredient.

The term “portion” refers to mini-tablet, tablet, pellet, bead, granule, a layer of a tablet, a coated layer, powder or any other known solid physical form prepared by standard methods known to the person skilled in the art, including but not limited to compression, granulation, spray coating, prilling, and extrusion/spheronization.

In an embodiment, the oral modified release pharmaceutical composition releases methylergonovine or a pharmaceutically acceptable salt thereof from the composition over a period ranging from about 0 hour to up to about 24 hours.

In a further embodiment, a portion of methylergonovine or a pharmaceutically acceptable salt thereof in the composition is released in a slow, continuous release manner and a remaining portion provides an initial load dose of methylergonovine or a pharmaceutically acceptable salt thereof.

In a further embodiment, the complete amount of methylergonovine or a pharmaceutically acceptable salt thereof in the composition, preferably in the extended release portion, is released in a slow, continuous release, without any initial load dose.

The oral pharmaceutical composition of the invention comprises up to 2 mg and preferably at least about 0.6 mg methylergonovine or a pharmaceutically acceptable salt thereof. The dose of methylergonovine in the composition may be about 0.6 mg, about 0.8 mg, about 1 mg, about 1.2 mg, about 1.4 mg, about 1.6 mg, about 1.8 mg or about 2 mg. The total dose may be provided as a single portion or multiple portions in the composition.

In an embodiment, the total dose of at least about 0.6 mg methylergonovine or a pharmaceutically acceptable salt thereof in the composition exhibits extended release.

In another embodiment, the composition comprises at least one portion of methylergonovine or a pharmaceutically acceptable salt thereof that exhibits extended release and at least one portion of methylergonovine or a pharmaceutically acceptable salt thereof in the composition that exhibits immediate release.

In a further embodiment, the composition comprises at least about 0.4 mg to about 1.6 mg dose of methylergonovine or a pharmaceutically acceptable salt thereof that exhibits extended release and at least about 0.2 mg to about 0.6 mg methylergonovine or a pharmaceutically acceptable salt thereof that exhibits immediate release.

The dose of methylergonovine or a pharmaceutically acceptable salt thereof in the immediate release portion may be about 0.2 mg, about 0.3 mg or about 0.4 mg.

The dose of methylergonovine or a pharmaceutically acceptable salt thereof in the extended release portion may be about 0.2 mg, about 0.3 mg, 0.4 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg or about 1.6 mg.

The oral pharmaceutical composition or modified release composition of the invention is preferably in the form of a solid dosage form. Such dosage forms include a tablet, a coated tablet, a multilayer (e.g. bilayer and trilayer) tablet, a capsule, a caplet, granules, pellets, minitablets, powder, and granules, pellets or powder filled into a capsule.

In an embodiment, the oral pharmaceutical composition of methylergonovine or a pharmaceutically acceptable salt thereof comprises:
(a) at least one immediate release portion comprising least about 0.2 mg to about 0.6 mg methylergonovine or a pharmaceutically acceptable salt thereof, and
(b) at least one extended release portion comprising about 0.4 mg to about 1.6 mg methylergonovine or a pharmaceutically acceptable salt thereof.

In another embodiment, the modified release oral pharmaceutical composition of methylergonovine or a pharmaceutically acceptable salt thereof comprises:
(a) a core comprising at least about 0.4 mg to about 1.6 mg methylergonovine or a pharmaceutically acceptable salt thereof exhibiting extended release, and
(b) at least one layer surrounding the core comprising least about 0.2 mg to about 0.6 mg methylergonovine or a pharmaceutically acceptable salt thereof exhibiting immediate release.

In another embodiment, the modified release oral pharmaceutical composition of methylergonovine or a pharmaceutically acceptable salt thereof comprises:
(a) at least one layer comprising least about 0.2 mg to about 0.6 mg methylergonovine or a pharmaceutically acceptable salt thereof exhibiting immediate release, and
(b) at least one layer comprising about 0.4 mg to about 1.6 mg methylergonovine or a pharmaceutically acceptable salt thereof exhibiting extended release.

The inventors further observed that the oral pharmaceutical composition of the invention provides release of methylergonovine over a period of up to about 24 hours. Such release profile is particularly desirable for patients who require round-the-clock therapeutic benefit, especially for patients suffering from migraine and refractive migraine.

In an embodiment, the immediate release portion, if present in the composition, exhibits complete release of methylergonovine or a pharmaceutically acceptable salt thereof within 1 hour after oral administration.

In another embodiment, the extended release portion of the composition exhibits release of methylergonovine or a pharmaceutically acceptable salt thereof over a period of up to about 24 hours after oral administration.

In a further embodiment, release of methylergonovine or pharmaceutically acceptable salt thereof from the extended release portion of the composition starts about 2 hours, about 4 hours, about 6 hours or about 8 hours after oral administration.

The invention further provides a method of treating various conditions responsive to methylergonovine, including a condition selected from migraine, refractory migraine, uterine atony, uterine haemorrhage, subinvolution of the uterus, or uterine haemorrhage in the second stage of labor.

In an embodiment, the method of treating migraine, refractory migraine, uterine atony, uterine haemorrhage, subinvolution of the uterus, or uterine haemorrhage in the second stage of labor comprises once daily oral administration of the pharmaceutical composition comprising at least about 0.6 mg dose of methylergonovine or pharmaceutically acceptable salt thereof as substantially described throughout the specification.

In a preferred embodiment, the method of treating refractory migraine comprises once daily oral administration of the pharmaceutical composition comprising at least about 0.6 mg dose of methylergonovine or pharmaceutically acceptable salt thereof as substantially described throughout the specification.

The modified release formulations of methylergonovine exhibit an IR profile, or a XR profile, or a combination of a XR profile with an IR profile. In some embodiments, the formulations may exhibit a pulsatile release profile. These specific release profiles are achieved by formulating methylergonovine with at least one of a release rate controlling compound and at least one excipient in a variety of inventive formulations.

The release rate controlling compounds of the current invention may be selected from hydrophilic rate controlling compounds and hydrophobic rate controlling compounds. The following non-limiting examples of such compounds are provided below.

Hydrophilic compounds may include one or more of the following: hydroxypropyl cellulose, hypromellose (hydroxypropyl methyl cellulose), methyl cellulose, polyethylene oxide, acacia, acrylic acid derivatives, alginic acid (and its salts and derivatives thereof), hydroxyethyl cellulose, povidone, carrageenan, carboxymethylcellulose, tragacanth, polyvinyl alcohol, xanthan gum and combinations thereof.

Hydrophobic compounds may include one or more of the following: ethyl cellulose, cellulose acetate, cellulose acetate butyrate, waxes (e.g., carnauba wax, microcrystalline wax), hydrogenated vegetable oils, Compritol 888 ATO (glyceryl behenate), Precirol ATO 5 (glyceryl palmitostearate), PEG glyceryl esters such as Gelucire 50/1, EUDRAGIT® NE 30 D (ethyl acrylate and methyl methacrylate copolymer), EUDRAGIT® RS (ammonio methacrylate copolymer, Type B) and EUDRAGIT® RL (ammonio methacrylate copolymer, Type A) polyvinyl acetate, cellulose acetate propionate, and combinations thereof.

Compounds that can be used as release rate controlling coatings may include one or more of the following: cellulose esters, cellulose acetate, cellulose acetate butyrate, ethyl cellulose, EUDRAGIT® NE 30 D (ethyl acrylate and methyl methacrylate copolymer), EUDRAGIT® RS (ammonio methacrylate copolymer, Type B) and EUDRAGIT® RL (ammonio methacrylate copolymer Type, A), ethyl acrylate methyl methacrylate copolymer, polyvinyl acetate, shellac, zein and combinations thereof.

The application of a release rate controlling compound coating is achieved using standard coating techniques such as spraying, dipping, casting, coating solvent evaporation, molding or compression coating.

The release rate controlling compounds described above may be used to prepare a variety of modified release systems, including:

(A) Matrix Systems—wherein an active pharmaceutical ingredient (methylergonovine), at least one release rate controlling compound, and at least one pharmaceutically acceptable excipient are homogeneously intermixed to form a matrix. Hydrophilic and hydrophobic compounds listed above may be used to prepare these methylergonovine-containing matrices. These matrices may be presented in the form of matrix tablets, matrix multiparticulates, or in the form of a layer coated onto a substrate.

Matrix tablets may be in the form of multiple layer tablets (e.g., bilayer or tri-layer tablets), tablet within a tablet, encapsulated mini-tablets or a tablet of compressed modified release particles. These matrix systems may be coated with release rate controlling compounds to add additional release rate controlling characteristics or a delayed release characteristics to the extended release profile of a formulation.

(B) Drug-Layered Systems—that comprise an inert core and at least one drug-containing layer coated onto this core. The drug containing layer(s) may be further coated with a layer of a release rate controlling compound selected from those listed above. If the drug-containing layer of the drug-layered system does not contain any release rate controlling compounds and is of an immediate release nature, then a release rate controlling coating is necessary for achieving the modified profiles of the current invention.

In cases where the drug-containing layer is an extended release matrix layer described above, the release rate controlling coating is optional and allows for additional modification of the release profile. For example, the coating may be used to modulate the release (slow initially, faster later; or fast initially, slower later), or to provide a delay in the release. In particular the release rate controlling coatings can include: cellulose esters, cellulose acetate, cellulose acetate butyrate, ethyl cellulose, EUDRAGIT® NE 30 D (ethyl acrylate and methyl methacrylate copolymer), EUDRAGIT® RS (ammonio methacrylate copolymer, Type B) and EUDRAGIT® RL (ammonio methacrylate copolymer, Type A), ethyl acrylate methyl methacrylate copolymer, polyvinyl acetate, cellulose acetate propionate, shellac, zein and combinations thereof.

In some embodiments of the invention, a core may not be inert but compositionally be of pure drug substance or a mixture of the drug substance and one or more pharmaceutically acceptable excipient producing an IR core. In such a case, the cores can undergo further processing as described above for inert cores to produce the desired extended release formulation.

Processes that may be used to produce formulations of this embodiment comprising a drug-containing core include solution or dry powder drug layering, compression coating, hot melt coating, supercritical fluid coating, electrostatic spray coating, agglomeration, granulation, pelletization, roller compaction, tablet compression, wet granulation with extrusion and spheronization, hot melt extrusion, and injection molding. Roller compaction, tablet compression, and the extrusion with spheronization processes are particularly helpful for the manufacturing of formulations with a high drug load.

Without putting any limitations thereon, exemplary formulations of the present invention having different modified pharmacokinetic (PK) profiles for methylergonovine are as follows.
a. There may be mixed IR and XR particles in a capsule, compressed tablet or any other dosage form (IR/XR mixed particles). The IR particles provide the initial release of the therapeutic agent followed by extended release from the XR particles (IR/XR mixed population of particles).
b. There may be a single population of particles in a capsule, compressed tablet or any other dosage form where the particles are either matrix XR particles, or IR cores further comprising an XR coating.
c. There may be mixed particles in a capsule, compressed tablet or any other dosage form where XR particles of differing drug release characteristics are combined.
d. There may be mixed particles in a capsule, compressed tablet or any other dosage form where delayed release (DR) particles of differing drug release characteristics are combined, optionally resulting in a pulsatile profile.
e. There may be mixed particles in a capsule, compressed tablet or any other dosage form where IR particles are mixed with DR particles (IR/DR mixed particles). The IR particles provide the initial release of the therapeutic agent followed by release from the DR particles resulting in pulsed PK profiles (IR/DR mixed population of particles).
f. There may be a single population of particles in a capsule, compressed tablet or any other dosage form where the pellet incorporates an IR core coated with DR coat, which is further coated with an IR drug layer. The outer IR drug layer provides an immediate release of the therapeutic agent followed by a delayed release from the DR core resulting in pulsed PK profile (IR/DR single population of particles).
g. There may be mixed particles in a capsule, compressed tablet or any other dosage form where IR particles are mixed with DR coated XR particles (IR/DR-XR). The IR particles provide the initial release of the therapeutic agent followed by delayed and extended release from the DR coated XR particles (IR/DR-XR mixed population of particles).
h. There may be a single population of particles in a capsule, compressed tablet or any other dosage form where the pellet incorporates an IR core coated with a XR coat, which is coated with a DR coat that is subsequently drug layered. The outer drug layer provides the initial immediate release of the therapeutic agent followed by delayed and extended release from the remainder of the pellet (IR/DR-XR single population of particles).
i. There may be mixed particles in a capsule, compressed tablet or any other dosage form where XR particles are mixed with DR particles. The XR particles provide the initial and continuing release of the therapeutic agent followed by release from the DR particles (XR/DR mixed population of particles). A single population of particles in a capsule, compressed tablet or any other dosage form where the pellet incorporates an IR core coated with a DR coat which is then coated with a drug layer that is subsequently coated with an XR coat to produce a fast XR layer. The fast XR outer layer provides the initial release of the therapeutic agent followed by delayed release from the DR core (XR-f/DR single population of particles).
j. There may be a XR tablet, which is either a matrix tablet or an XR-coated tablet.
k. There may be a DR tablet coated with an IR drug layer.
l. There may be one, or more than one, DR tablets mixed with one or more IR tablets in a capsule.
m. There may be a XR tablet coated with a DR coat, then coated with an IR drug layer.
n. There may be a bi-layer tablet with one layer containing the drug in XR form and a second layer containing the drug in an IR form.
o. There may be a bi-layer tablet with one layer containing the drug in XR form and a second layer containing the drug in DR form.
p. There may be a DR coated matrix tablet providing a DR/XR profile.

(C) Osmotic Release Systems—in a further embodiment, the invention provides an extended release methylergonovine preparation in the form of an osmotic tablet, wherein the drug release rate is determined by the rate of water permeation into the tablet core through a semi-permeable membrane coating.

For the preparation of an osmotic tablet, methylergonovine may be mixed with osmotic agent(s), tableting aides such as diluents and lubricants, and other commonly used excipients. The mixture is tableted either by direct compression or granulation followed by compression. Tablets are then coated with at least one release rate controlling compound that forms a semi-permeable membrane that surrounds each tablet.

The semipermeable membrane, which surrounds the drug-containing core, comprises at least one release rate controlling compound selected from cellulose esters, cellulose ethers and cellulose ester ethers. Non-limiting examples of such compounds include cellulose acylate, cellulose ethyl ether, cellulose diacylate, cellulose triacylate, cellulose acetate, cellulose diacetate, cellulose triacetate, mono-, di- and tricellulose alkyls, mono-, di- and tricellulose aroyls, and combinations thereof. Additional release rate controlling compounds include ethyl cellulose, EUDRAGIT® NE 30 D (ethyl acrylate and methyl methacrylate copolymer), EUDRAGIT® RS (ammonio methacrylate copolymer, Type B) and EUDRAGIT® RL (ammonio methacrylate copolymer, Type A), ethyl acrylate methyl methacrylate copolymer.

The semi-permeable membrane may be applied on the tablets using standard coating techniques such as spraying, dipping, casting, coating solvent evaporation, molding or compression coating. An orifice is then drilled in the tablet coat using laser tablet drilling system or other mechanical means to allow the release of drug from the core.

Osmotic agents used for the practice of the current invention are well known in the art and include non-swellable compounds represented by, but not limited to polyols, carbohydrates (including monosaccharides, oligosaccharides, polysaccharides and sugar alcohols), acids, salts and hydrophilic compounds. For example, osmotic agents may be selected from mannitol, maltrin, xylitol, maltitol, lactitol, isomalt, sorbitol, arabitol, erythritol, ribitol, insositol, trehalose, lactose, glucose, sucrose, raffinose, fructose, dextran, glycine, urea, citric acid, tartaric acid, ascorbic acid, aspartame, malic acid, sodium chloride, potassium chloride, magnesium chloride, disodium hydrogen phosphate, sodium phosphate, potassium phosphate, sodium sulfate, lithium sulfate, magnesium sulfate, magnesium succinate, sodium bicarbonate, sodium carbonate, sodium acetate, sodium ascorbate, polyethylene glycol, maltodextrin, cyclodextrins and derivatives, non-swelling block polymers of PEO and PPO, polyethylene glycols, cellulose ethers, and combinations thereof.

Osmotic tablets can be formulated as a single or as a multiple layer core. In one embodiment, the osmotic tablet comprises a bilayer core, wherein one layer comprises agents to modulate drug release, such as a solubilizer, that are released in an extended manner, and the second layer comprises the drug and potentially other agents to modulate drug release.

An overcoat of drug can be applied to the tablet following a functional coating to provide an immediate release component to the dosage form. Alternatively, the osmotic tablet may be coated with an enteric compound on top of the semipermeable membrane providing a DR/XR profile.

In addition to the release rate controlling compounds, a number of pharmaceutically acceptable excipients may be used in the formulations of the invention as disclosed above. These excipients are well known in the art, and include binders and diluents, such as povidone, starch, gelatin, maltodextrin, methylcellulose, hydroxypropyl cellulose, hydroxypropyl methylcellulose, carboxymethylcellulose, sucrose, dextrose, acacia, tragacanth and locust bean gum, microcrystalline cellulose, dicalcium phosphate, calcium sulfate, cellulose, and talc; lubricants such as sodium stearyl fumarate and the metallic stearates such as magnesium stearate; wetting and solubilizing agents such as sodium docusate, sodium lauryl sulfate, polyethylene glycol, lecithin, poloxamer, polysorbates, polyoxyethylene ethers and sorbitan esters; disintegrants such as crosslinked sodium carboxymethylcellulose, sodium starch glycolate and crospovidone; buffering agents and/or pH modulating agents, such as aluminum hydroxide, ammonium bicarbonate, ammonium carbonate, ammonium phosphate, arginine, calcium acetate, calcium ascorbate, magnesium acetate, magnesium carbonate, potassium acetate, potassium bicarbonate, potassium carbonate, potassium phosphate dibasic, potassium sodium tartrate, potassium citrate, sodium citrate, sodium phosphate monobasic, sodium phosphate dibasic, sodium phosphate tribasic, sodium acetate, sodium bicarbonate, sodium ascorbate, sodium carbonate, fumaric acid, malic acid, tartaric acid, ascorbic acid, aspartic acid, alginic acid, glutamic acid, sorbic acid, and succinic acid; and glidants such as talc, starch and colloidal silicon dioxide; pore formers modulating the permeability of the semipermeable rate controlling membrane such as povidone, hypromellose, hydroxyethyl cellulose, hydroxypropyl cellulose, organic acids and salts amongst other excipients.

(D) Gastro-Retentive Systems—in a further embodiment, the invention provides an extended release methylergonovine preparation in the form of a gastro-retentive tablet, in particular a gastro-retentive extended release tablet. The gastro-retentive tablet is designed to be retained in the stomach for up to 6 hours after ingestion, after which the remaining dosage form and essentially all undissolved drug is released into the duodenum to transit through the gastrointestinal tract. The in-vitro dissolution profile used for the GR-ER tablet releases 80% of the dose of drug contained in the dosage form in approximately 10 hours.

### EXAMPLES

### Example 1

### Composition of Methylergonovine Modified Release Matrix Tablet, 0.4 mg

| Item No. | Component | mg/Tablet |
|---|---|---|
| 1 | Methylergonovine Maleate, USP | 0.40 |
| 2 | Povidone | 3.00 |
| 3 | Gelatin | 1.00 |
| 4 | Acacia | 1.00 |
| 5 | Tartaric Acid | 0.60 |
| 6 | Methylparaben | 0.04 |
| 7 | Propylparaben | 0.01 |
| 8 | Corn Starch | 5.00 |
| 9 | Microcrystalline Cellulose | 20.00 |
| 10 | Lactose Monohydrate | 42.00 |
| 11 | Hypromellose (METHOCEL ® K15M) | 22.75 |
| 12 | Stearic Acid | 4.00 |
| 13 | Colloidal Silicon Dioxide | 0.20 |
| 14 | Purified Water, USP | qs |
| | Total | 100.00 |

### Manufacturing Process:

The batch of Example 1 was manufactured by a wet granulation process. Lactose monohydrate, microcrystalline cellulose, corn starch, and povidone were dry mixed in a granulator and then was granulated with a solution containing methylergonovine maleate, gelatin, acacia, povidone, methylparaben and propylparaben dissolved in purified water. The wet granulation was oven dried at 40° C. to a moisture level of less than 1% (w/w) and then sized by passing through an 18 mesh sieve. The dried granules were then milled in a Fitzmill and blended with in a V-blender with hypromellose (METHOCEL® K15M), stearic acid and colloidal silicone dioxide. The final blend was compressed into modified-release tablets at the dose strength of 0.4 mg.

### Example 2

### Composition of Methylergonovine Modified Release Matrix Tablet, 0.8 mg

| Item No. | Component | mg/Tablet |
|---|---|---|
| 1 | Methylergonovine Maleate, USP | 0.80 |
| 2 | Povidone | 3.00 |
| 3 | Gelatin | 1.00 |
| 4 | Acacia | 1.00 |
| 5 | Tartaric Acid | 1.60 |
| 6 | Methylparaben | 0.04 |
| 7 | Propylparaben | 0.01 |
| 8 | Corn Starch | 5.00 |
| 9 | Microcrystalline Cellulose | 20.00 |
| 10 | Lactose Monohydrate | 32.00 |
| 11 | Hypromellose (METHOCEL ® K15M) | 31.35 |
| 12 | Stearic Acid | 4.00 |
| 13 | Colloidal Silicon Dioxide | 0.20 |
| 14 | Purified Water, USP | qs |
| | Total | 100.00 |

### Manufacturing Process:

The batch of Example 2 was manufactured by a wet granulation process. Lactose monohydrate, microcrystalline cellulose, corn starch, and povidone were dry mixed in a granulator and then was granulated with a solution containing methylergonovine maleate, gelatin, acacia, povidone, methylparaben and propylparaben dissolved in purified water. The wet granulation was oven dried at 40° C. to a moisture level of less than 1% (w/w) and then sized by passing through an 18 mesh sieve. The dried granules were then milled in Fitzmill and blended with in a V-blender with hypromellose (METHOCEL® K15M), stearic acid and colloidal silicone dioxide. The final blend was compressed into modified-release tablets at the dose strength of 0.4 mg.

### Example 3

### Composition of Methylergonovine Immediate Release Matrix Tablet, 0.2 mg

| Item No. | Component | mg/Tablet |
|---|---|---|
| 1 | Methylergonovine Maleate, USP | 0.20 |
| 2 | Povidone | 3.00 |
| 3 | Gelatin | 1.00 |
| 4 | Acacia | 1.00 |
| 5 | Tartaric Acid | 0.60 |
| 6 | Methylparaben | 0.04 |
| 7 | Propylparaben | 0.01 |
| 8 | Corn Starch | 5.00 |
| 9 | Microcrystalline Cellulose | 43.15 |
| 10 | Lactose Monohydrate | 42.00 |
| 11 | Stearic Acid | 4.00 |
| 12 | Purified Water, USP | qs |
| | Total | 100.00 |

### Manufacturing Process:

The batch of Example 3 was manufactured by a wet granulation process. Lactose monohydrate, microcrystalline cellulose, corn starch, and povidone were dry mixed in a granulator and then was granulated with a solution containing methylergonovine maleate, gelatin, acacia, povidone, methylparaben and propylparaben dissolved in purified water. The wet granulation was oven dried at 40° C. to a moisture level of less than 1% (w/w) and then sized by passing through an 18 mesh sieve. The dried granules were then milled in Fitzmill and blended within a V-blender with stearic acid to yield the immediate-release blend

### Example 4

### Composition of Methylergonovine Bilayer Extended Release Matrix Tablet, 0.6 mg

The immediate release blend from Example 3 and the extended release blend from Example 1 were compressed into bilayer extended release tablets.

### Example 5

### Composition of Immediate Release Methylergonovine Layered and Coated Beads

| Item No. | Component | mg/Capsule |
|---|---|---|
| 1 | Methylergonovine Maleate, USP | 0.20 |
| 2 | Povidone, USP | 3.00 |
| 3 | Gelatin, NF | 1.00 |
| 4 | Acacia, NF | 1.00 |
| 5 | Tartaric Acid, NF | 0.60 |
| 6 | Methylparaben, NF | 0.04 |
| 7 | Propylparaben, NF | 0.01 |
| 8 | Sugar Spheres, NF 30/35 mesh | 80.14 |
| 9 | Talc, USP | 4.00 |
| | Kollicoat ® | 10.00 |
| 10 | Purified Water, USP | qs |
| | Total | 100.00 |

### Manufacturing Process:

The drug layering dispersion of Example 5 was prepared by dissolving the methylergonovine maleate, gelatin, acacia, povidone, methylparaben and propylparaben in purified water, then dispersing the talc, and stirring for 20 minutes. The drug dispersion was then applied onto the 30/35-mesh sugar spheres in a Glatt GPCG-1 fluidized bed coater while being stirred. The inlet temperature ranged between 50-55° C. to attain product at a temperature of 40-42° C. The dispersion was sprayed at the rate of 8-12 g/min with atomization air pressure of 1.5 bar. Optionally, the drug layered beads were subsequently applied with a polyvinyl alcohol-polyethylene glycol grafted copolymer (Kollicoat®) coating solution at 10% coating level and subsequently dried. The uncoated beads (100 mg) were filled into a capsule to yield immediate-release pellets in a capsule

### Example 6

### Composition of Immediate Release Methylergonovine Microtablets in Capsule, 0.2 mg

| Item No. | Component | mg/Tablet |
|---|---|---|
| 1 | Methylergonovine Maleate, USP | 0.20 |
| 2 | Povidone, USP | 3.00 |
| 3 | Gelatin, NF | 1.00 |
| 4 | Acacia, NF | 1.00 |
| 5 | Tartaric Acid, NF | 0.60 |
| 6 | Methylparaben, NF | 0.04 |
| 7 | Propylparaben, NF | 0.01 |
| 8 | Lactose Monohydreate, NF | 42.00 |
| 9 | Microcrystalline Cellulose, NF | 43.15 |
| 10 | Corn Starch, NF | 5.00 |
| 11 | Stearic Acid, NF | 4.00 |
| 12 | Purified Water, USP | qs |
| | Total | 100.00 |

### Manufacturing Process:

The drug layering solution of Example 6 was prepared by dissolving the methylergonovine maleate, gelatin, acacia, povidone, methylparaben and propylparaben in purified water. The drug solution was then sprayed onto a blend of lactose monohydrate, microcrystalline cellulose, corn starch and granulated using the wet granulation process. The wet granules were dried in an oven. The dried granules were them milled in Fitzmill and blended in a V-blender with stearic acid. The final blend was compressed into 2 mm microtablets using multipump tooling. Microtablets were subsequently coated with the polyvinyl alcohol-polyethylene glycol grafted copolymer (Kollicoat®) coating solution at 10% coating level and subsequently dried. 110 mg of these coated microtablets filled into the capsule to yield immediate release microtablets in a capsule.

### Example 7

### Composition of Multiparticulate-Based, Modified Release Methylergonovine Capsule, 0.8 mg

The dried pellets from Example 5 were screened (stacked 20 mesh over 40 mesh sieves) and then seal coated using a Wurster process (GPCG-1) to a 5% weight gain with Opadry® II White. Following application of the seal coat, pellets were then coated on the GPCG-1 with Surelease® E-7-19010 to a weight gain of 15% (w/w). The coated pellets were oven cured for 24 hours at 50° C. Appropriate portions of extended release coated beads were then blended with immediate release beads from Example 5 and filled into hard gelatin capsules.

### Example 8

### Composition of Microtablets-Based, Modified Release Methylergonovine Capsule, 0.6 mg

The compressed microtablets from Example 6 were seal coated using a Wurster process (GPCG-1) to 5% weight gain with Opadry® II White. Following application of the seal coat, pellets were then coated on the GPCG-1 with Surelease® E-7-19010 to a weight gain of 10% (w/w). The coated microtablets were oven cured for 24 hours at 50° C. Next, 300 mg of modified-release coated microtablets were then blended with immediate-release beads from Example 5 in appropriate portions and filled into hard gelatin capsules.

### Example 9

### Composition of Multiparticulates-Based, Modified Release Methylergonovine Capsule

Immediate release beads from Example 5 were initially seal-coated with Hydroxypropyl Methylcellulose (Opadry®) at 6% weight gain and subsequently applied with dispersions of EUDRAGIT® RS (ammonio methacrylate copolymer Type B) and EUDRAGIT® RL (ammonio methacrylate copolymer Type A) polymers containing triethyl citrate and talc (anti-tacking agent) in various proportions and various coating levels and finally seal-coated with hydroxypropyl methylcellulose (Opadry®) at a 6% weight gain. The coated beads were encapsulated in hard gelatin capsules.

What is claimed is:

1. An oral modified release pharmaceutical composition configured for once daily administration comprising at least about 0.6 mg methylergonovine or a pharmaceutically acceptable salt thereof.

2. The modified release composition of claim 1, wherein said composition provides release of methylergonovine or a pharmaceutically acceptable salt thereof over a period ranging from more than 0 hours to up to 24 hours.

3. The modified release composition of claim 1, wherein said composition provides extended release of methylergonovine or a pharmaceutically acceptable salt thereof, with or without an initial load dose.

4. The modified release composition of claim 3, wherein said composition comprise:
(a) at least one extended release portion comprising methylergonovine or a pharmaceutically acceptable salt thereof, and
(b) optionally, at least one immediate release portion comprising methylergonovine or a pharmaceutically acceptable salt thereof.

5. The modified release composition of claim 4, wherein the extended release portion exhibits release of methylergonovine or a pharmaceutically acceptable salt thereof over a period of up to 24 hours after oral administration.

6. The modified release composition of claim 4, wherein the extended release portion comprises at least about 0.4 mg to about 1.6 mg methylergonovine or a pharmaceutically acceptable salt thereof.

7. The modified release composition of claim 4, wherein said immediate release portion comprises at least about 0.2 mg to about 0.6 mg methylergonovine or a pharmaceutically acceptable salt thereof.

8. The modified release composition of claim 1, wherein said composition is a solid oral dosage form selected from a tablet, a multilayer tablet, a capsule, a caplet, granules, pellets, powder, and granules, pellets or powder filled in a capsule.

9. The modified release composition of claim 1, wherein the pharmaceutically acceptable salt of methylergonovine is maleate.

10. The modified release composition of claim 1, wherein the pharmaceutically acceptable salt of methylergonovine is tartrate.

11. A modified release oral composition configured for once daily administration comprising:
(a) a core comprising at least about 0.4 mg to about 1.6 mg methylergonovine or a pharmaceutically acceptable salt thereof exhibiting extended release, and
(b) at least one layer surrounding the core comprising at least about 0.2 mg to about 0.6 mg methylergonovine or a pharmaceutically acceptable salt thereof exhibiting immediate release.

12. A solid oral unit dosage form configured for once daily administration comprising:
(a) at least one layer comprising least about 0.2 mg to about 0.6 mg methylergonovine or a pharmaceutically acceptable salt thereof exhibiting immediate release, and
(b) at least one layer comprising about 0.4 mg to about 1.6 mg methylergonovine or a pharmaceutically acceptable salt thereof exhibiting extended release.

13. A method for the treatment of a methylergonovine responsive condition selected from migraine, refractory migraine, uterine atony, uterine haemorrhage, subinvolution of the uterus, or uterine haemorrhage in the second stage of labor, said method comprising once daily oral administration of the modified release composition of claim 1.

14. The method of claim 16, wherein said methylergonovine responsive condition is refractory migraine.
